(19) United States
(12) Patent Application Publication
(10) Pub. No.: US 2019/0008879 A1
(43) Pub. Date: Jan. 10, 2019

(54) TREATMENT FOR CANCER BY A COMBINATION OF AN INOSITOL PHOSPHORIC DERIVATIVE AND MAGNESIUM CHLORIDE

(71) Applicant: Guy PARC, RUEIL-MALMAISON (FR)

(72) Inventor: Guy PARC, RUEIL-MALMAISON (FR)

(21) Appl. No.: 16/026,832

(22) Filed: Jul. 3, 2018

(30) Foreign Application Priority Data
Jul. 10, 2017 (FR) 1756494

(51) Int. Cl. A61K 31/6615; A61K 47/12; A61K 9/08; A61K 9/14; A61P 35/00

(52) U.S. Cl. CPC A61K 31/6615; A61K 47/12; A61K 9/08; A61K 33/14; A61P 35/00; A61K 2300/00; A61K 9/14

(57) ABSTRACT

Disclosed is a pharmaceutical composition including magnesium chloride and at least one compound selected from inositol hexaphosphoric acid, inositol pentaphosphoric acids, inositol tetraphosphoric acids or one of their salts, in association with one or more pharmaceutically acceptable excipients and/or vehicles, and their use in the treatment and/or prevention of cancer.

### BACKGROUND OF THE INVENTION

### Field of the Invention

The present invention refers to the field of the therapeutic treatment for cancers in humans or animals. The invention particularly relates to the use in human or veterinary medicine of a composition for its anticancer activity.

### Description of the Related Art

According to recent statistics of the World Health Organization (WHO), cancer represents the second leading cause of death after cardiovascular diseases in industrialized countries. The means implemented to deal with cancer diseases involve enhancing early diagnosis, and also improving drug treatments. The discovery of new molecules or original compositions, the tumor cell specificity of which would be complete relative to healthy cells, enables the development of new therapies.

Before making a therapeutic choice for cancer treatment, a number of parameters have to be studied: type of cancer (sarcoma, melanoma . . . ), affected organ, stage of cancer progression, all of the prognostic factors and patient specific characteristics (age, general health, mental state . . . ). From these data, a therapy can be chosen, either local or general. The most efficient treatments are local therapies involving surgery and/or radiotherapy methods. They treat less developed injuries and cure most of localized cancers. General therapies, chemotherapy and/or hormonotherapy are generally palliative or adjuvant treatments. These treatments are implemented in the case of localized but more developed cancers. They make it possible to cure a limited number of generalized cancers but improve the life expectancy of patients (cf. Capdeville R., Buchdunger E., Zimmermann J., Matter A., Nature Rev. Drug Discov., 2002, 1, 493; Eisenberg B. L., Von Mehren M., Expert Opin Pharmacother., 2003, 6, 869; Gilman A., Philips F. S., Science, 1946, 103, 409; Gingras D, Béliveau R. Med. Sci., 1997, 13, 1428-35.

The restriction on the use of known anticancer agents concerns their high toxicity which causes a large number of side effects and can even result to the patient death. The chemical weapons used in treating cancer are supposed to destroy cancer cells while sparing healthy ones. But selectivity is quite relative, and most of the drugs used in chemotherapy have a significant hematologic toxicity. Reducing adverse side effects, especially those with medically and psychologically serious consequences, is as important as trying to improve the effectiveness of a given drug. Overall, the current arsenal of the chemotherapist still consists of old, highly cytotoxic drugs, most known anticancer agents being already several decades old, very poorly targeted, at least at the cell level, and offering no alternatives to the resistance phenomena. Thus, there is a need for new anticancer compositions for use in chemotherapy, which ideally target only cancer cells.

It was during his long stay in Africa, in the 70's, that Doctor Burkitt had noticed a correlation between a diet particularly rich in whole-grain cereals and thus in fibers and the low incidence of colorectal, breast, prostate and endometrial cancer as compared with that of Europeans (Burkitt D. P., Dietary fiber and Cancer, J. Nut. 1988 118: 531-533). In its update published in 2015 («Nutrition et Prévention Primaire des Cancers; Actualisation des Données», Jun. 2015), the Institut National du Cancer (INCA) recognizes a convincing correlation between the fiber intake and the reduction in the incidence of colorectal cancer and a likely correlation with breast cancer.

By 1985, American researchers Graf and Eaton showed that it is actually the phytic acid salts in cereal fibers that cause the antitumor effect and not the fibers themselves (Graf E., Eaton J. W., Dietary suppression of colonic cancer: Fiber or Phytate?, Cancer 1985, 56, 717-718). The phytic acid compounds have long been considered as antinutritional factors in human food as they limit the absorption of certain essential minerals such as calcium, magnesium and iron at the gastrointestinal tract.

The phytic acid (CAS no: 83-86-3), also known as myo-inositol hexaphosphoric acid, is a biomolecule present as phytates associated with different cations in oleaginous fruits, pollen, spores and in the seeds of many cereals and legumes where it represents up to 90% of the total phosphorus reserves. Phytic acid is mainly found in the aleurone layer or the embryo of seeds.

Phytic acid can be extracted from plant matrices as a phytin. The term “phytin” refers to phytic acid salts (or phytates) which essentially comprise as the counterions Mg²⁺, Ca²⁺and/or K+. Phytin is generally found as a calcium and magnesium mixed salt of phytic acid.

However, recent studies showed that phytic acid and phytin can have a wide range of beneficial effects on human health (for review, Bohn et al., Journal of Zhejang university Science B, 2008, 9(3): 165-191).

Phytic acid and phytin can thus participate in the lowering of cholesterol level, normalization of glycemia and stimulation of the immune system. These compounds can also be used to prevent nephrolithiasis, skin hyperpigmentation but also cell premature ageing through their antioxidant activity.

It was also shown that phytic acid is able to inhibit the key processes involved in carcinogenesis such as abnormal cell proliferation and angiogenesis. The phytic acid can also induce apoptosis and participate in the cell immune response stimulation.

Thus, the antitumor properties of phytin were demonstrated by a lot of pre-clinical trials showing a real antiproliferative activity on many tumor stem cells at median lethal doses LD50 of from 200 μM to 2.5 mM and on animal models (for review, Vucenik I., Shamsuddine A. M., Cancer inhibition by inositol hexaphosphate (IP6) and inositol: from laboratory to clinic, Journal of nutrition 2003, 133: 3778S-3874S.

The publication of Bacic I., Druzijanic N., Karlo R., Skific I., Jagic S., Efficacy of IP6+ inositol in the treatment of breast cancer patients receiving chemotherapy: prospective, randomized, pilot clinical study, Journal of Experimental & Clinical Cancer Research 2010, 29:12, describes a small-scale clinical trial carried out during six month on 14 patients with invasive ductal breast cancer. Administration of phytin at high dosage (6 g/d), in addition to a chemotherapy, has preserved from cytopenia (leucocyte and platelet quantitative deficiency), while improving quality of life (reduced side effects), but has no impact on tumor progression.

The European patent EP 2452577 discloses the use of phytin in combination with myo-inositol and an ascorbic acid compound for preventing cancers and stimulating the immune system. Indeed, it was shown that the combination of phytin with myo-inositol is able to modulate some biological processes involved in the carcinogenesis such as abnormal cell proliferation and angiogenesis, and it is suggested that phytic acid and vitamin C work synergistically to boost the immune response and prevent the oxidative stress and therefore, the cellular ageing.

Accordingly, phytic acid and phytin are regarded as particularly valuable agents for the treatment but also for the prevention of cancer. The pentaphosphoric and tetraphosphoric derivatives of inositol are also known to have antitumor properties, the myo-inositol 1,3,4,5,6 pentakis phosphate being even described as having an antitumor activity higher than that of phytin (Maffucci T. et al., Cancer Res. 2005, 65, 8339-49: anti-angiogenic action by inhibiting the PI3K/Akt pathway at a concentration of 50 μM).

### SUMMARY OF THE INVENTION

The object of the invention is a pharmaceutical composition comprising magnesium chloride and at least a compound selected from inositol hexaphosphoric acid, inositol pentaphosphoric acids, inositol tetraphosphoric acids or one of their salts, in combination with one or more pharmaceutically acceptable excipients and/or vehicles.

This pharmaceutical composition is primarily intended to be used for therapeutic purposes, preferably for use in the treatment and/or the prevention of cancer in human or veterinary medicine.

Indeed, it was shown that the composition of the present invention has an in vivo antiproliferative activity in human against highly aggressive pancreatic tumors, and thus, it can be used for the treatment or the prevention of cancers. Moreover, the composition according to the invention is not cytotoxic in vivo against healthy cells at the dosages for which it has an anticancer activity.

Thus, the invention also relates to a therapeutic method comprising administering to a subject in need thereof an effective amount of the present pharmaceutical composition 1. Said method is preferably a method of treating and/or preventing cancer, such as pancreas cancer, breast cancer, lung cancer, glioblastoma multiforme, preferably pancreas cancer.

### DESCRIPTION OF THE PREFERRED EMBODIMENTS

Certain compounds of the invention can occur both in solvated and non-solvated form, for example in hydrated form. Generally, the solvated forms are equivalent to non-solvated forms and are included in the scope of the present invention. Certain compounds of the present invention can occur in many crystalline or amorphous forms. Generally, all the physical forms are equivalent for the intended uses by the present invention and are included in the scope of the present invention.

Some compounds used in the composition according to the invention have one or more asymmetric (optical) centers, such that stereoisomers (enantiomers or diastereoisomers) can exist. It is understood that the invention extends to all the enantiomers and diastereoisomers of these compounds and mixtures thereof, including racemates. In other words, the compounds used in the composition according to the invention can be used as a purified enantiomer or as an enantiomer mixture. The different isomers can be separated according to methods known to those skilled in the art, including by chiral chromatography, high performance liquid chromatography (HPLC) or by fractioned crystallization.

In the present application, reference is generally made to inositol hexaphosphoric acid and salts thereof, the preferred compounds according to the invention, but it is understood that all embodiments of the invention relating to the inositol hexaphosphoric acid and its salts can also be applied to the inositol pentaphosphoric acids and inositol tetraphosphoric acids or salts thereof.

The first essential component of the composition according to the invention is a compound selected from inositol hexaphosphoric acid, inositol pentaphosphoric acids, inositol tetraphosphoric acids or a salt thereof, preferably inositol hexaphosphoric acid or a salt thereof.

The inositol hexaphosphoric acid or a salt thereof is preferably present in the composition according to the invention in the form of its stereoisomer myo-inositol hexaphosphoric acid (phytic acid) or a salt thereof. It may also be present in the form of one or more other stereoisomers, including the scyllo-, muco-, D-chiro- and neo-stereoisomers.

The inositol pentaphosphoric acids (including the inositol 1,3,4,5,6-pentaphosphoric acid, preferably in the myo configuration) are naturally present in the bird red blood cells where they operate as oxygen release effectors. They may also be obtained by synthesis. They are preferably present in the composition according to the invention in the form of their stereoisomers myo-inositol pentaphosphoric acids. They may also be present in the form of one or more other stereoisomers, including the scyllo-, muco-, D-chiro- and neo-stereoisomers.

The inositol tetraphosphoric acids are preferably present in the composition according to the invention in the form of their stereoisomers myo-inositol tetraphosphoric acids. They may also be present in the form of one or more other stereoisomers, including the scyllo-, muco, D-chiro- and neo-stereoisomers. These compounds are preferably selected from inositol 1,3,4,5-tetraphosphoric acid and inositol 1,3,4,6-tetraphosphoric acid, both preferably in the myo configuration.

In the present application, reference is generally made to phytic acid and salts thereof (phytin), but it is understood that all of the embodiments of the invention relating to the phytic acid and salts thereof can be applied to the other stereoisomers of the inositol hexaphosphoric acid and its salts. It means that the expression “inositol hexaphosphoric acid” may be substituted for the expression “phytic acid” in all the particular embodiments of the invention. The same is true for the expressions “inositol hexaphosphoric acid and salts thereof” and “phytic compounds”, referring to the phytic acid and its salts, and the expressions “salt of the inositol hexaphosphoric acid” and “phytin”.

In the present invention, the inositol hexaphosphoric acid is preferentially used as one of its salts, preferably phytin.

Within the meaning of the present invention, a salt of the inositol hexaphosphoric acid comprises one or more counterions such as Ca²⁺, Mg²⁺, K⁺, Na⁺, Fe²⁺, Fe³⁺, Co²⁺, Zn²⁺and Cu²⁺. In a preferred embodiment, this salt corresponds to the basic forms of the inositol hexaphosphoric acid, preferably phytic acid (myo-inositol hexaphosphoric acid), associated primarily with Ca²⁺and/or Mg²⁺and/or K⁺cations, most preferably represents a calcium and magnesium mixed salt of phytic acid, i.e., phytin.

Phytin can be obtained from cereal seeds. Vegetable source suitable for the preparation of a phytin according to the invention includes wheat, corn and rice. As an example, phytin can be obtained from wheat bran, rice bran and corn seed embryo.

In some embodiments, a salt of inositol hexaphosphoric acid or a phytin obtained from cereal seeds and having a percentage by mass of calcium of from 0.05 to 20% and/or a percentage by mass of magnesium of from 3% to 18% is used, the percentages being expressed in relation to the total weight of phytin.

When the composition according to the invention is liquid, preferably aqueous, the inositol hexaphosphoric acid, the inositol pentaphosphoric acids, the inositol tetraphosphoric acids and salts thereof are present in the composition in an amount of from 70 g/L to 270 g/L, preferably from 80 g/L to 150 g/L, on the understanding that this amount is expressed in relation to the total volume of the composition. These amounts also apply individually to the inositol hexaphosphoric acid and its salts, the inositol pentaphosphoric acids and their salts and the inositol tetraphosphoric acids and their salts.

The other essential component of the composition according to the invention is the magnesium chloride. It can be used in anhydrous, hexahydrate form, or in other forms such as nigari, obtained from seawater.

Magnesium chloride can also be synthesized in situ by treatment of magnesium salt of inositol hexaphosphoric acid, inositol pentaphosphoric acids, or inositol tetraphosphoric acids with hydrochloric acid.

Typically, the inositol hexaphosphoric acid and the salts thereof on the one hand, and magnesium chloride on the other hand, are present in a mass ratio ranging from 4/1 to 16/1, preferably, from 6/1 to 12/1. When the composition according to the invention is liquid, the magnesium chloride is present so that its amount is in the range of from 5 g/L to 30 g/L. Preferably, the inositol hexaphosphoric acid, inositol pentaphosphoric acids, inositol tetraphosphoric acids or salts thereof, on the one hand, and the magnesium chloride, on the other hand (naturally counted as anhydrous salt), are present in a mass ratio ranging from 2/1 to 16/1, preferably, from 4/1 to 12/1, most preferably from 5/1 to 10/1.

A human or veterinary pharmaceutical composition according to the invention may also comprises one or more other active principles different from the inositol hexaphosphoric acid, inositol pentaphosphoric acids, inositol tetraphosphoric acids or a salt thereof and the magnesium chloride, especially to enhance its efficiency, including one or more other anticancer compounds. As other active principles that may be included in a pharmaceutical composition according to the invention, mention may be especially made of antihistaminic agents, anti-inflammatory agents, disinfecting agents or local anesthetic agents.

According to an embodiment of the invention, the composition according to the invention contains only, as anticancer active principle, inositol hexaphosphoric acid, inositol pentaphosphoric acids, inositol tetraphosphoric acids or a salt thereof, preferably, only inositol hexaphosphoric acid or a salt thereof.

According to another embodiment of the invention, the composition according to the invention contains an additional anticancer agent, such as taxane (for example paclitaxel or docetaxel), vinblastine, vinorelbine, vincristine, bleomycin, temozolomide, 5-fluorouracil and/or an angiogenesis inhibitor (for example bevacizumab).

Alternatively and additionally, the composition according to the invention can be used in combination with another therapeutic treatment, notably in combination with an additional anticancer treatment administered separately, such as a treatment with one of the above mentioned additional anticancer agents, targeting for example cancer cell proliferation and/or angiogenesis.

The composition may comprise at least one additional compound having a physiological or pharmaceutical interest. It may be a compound capable of acting in combination with phytic compounds, for example, a compound capable of stimulating immune defenses or even a compound having an action distinct from that of phytin. Examples of such compounds are vitamins and their precursors, amino acids, amino acid derivatives such as taurine, oligopeptides, fatty acids, hormones, flavonoids, plant extracts, inositol and mixtures thereof. These additional compounds are described in detail in the patent EP 2452577, on behalf of the applicant.

The additional compound(s) to be added in the composition can be determined on the basis of the desired physiological effects or the target audience. As an example, if a high antioxidant activity is required, a compound selected from vitamin C, vitamin E, quercetin or even a green tea extract could be, for example, associated with the inositol hexaphosphoric acid compounds.

The additional compound(s) for physiological or pharmaceutical purposes are typically present in the composition, when it is in liquid form, in an amount ranging from 0.01 g/L to 900 g/L, preferably, from 0.1 g/L to 400 g/L. The content of an additional compound is to be determined based on the nature of this compound and the desired effect. Additional compounds being commonly used in the preparation of pharmaceutical compositions, one of ordinary skill in the art can refers to the usual doses used in the state of the art to determine, for each additional compound, the concentration at which it must be introduced in the composition according to the present invention.

In a preferred embodiment according to the invention, the pharmaceutical composition further comprises inositol, preferably myo-inositol. Myo-inositol (CAS number: 87-89-8) corresponds to the dephosphorylated form of phytic acid and has only a moderate antitumor activity (Estensen, R. D., Wattenberg, L. W., Carcinogenesis 1993, 14 (9), 1975-1977). However, this compound works as a vitamin by having many beneficial effects improving the general condition of the patient. Typically, the inositol hexaphosphoric acid, inositol pentaphosphoric acids, inositol tetraphosphoric acids and salts thereof on the one hand, and the inositol on the other hand (preferably myo-inositol), are present in a mass ratio ranging from 1.5/1 to 8/1, preferably 2.5 to 6. When the composition according to the invention is liquid, the inositol is present in such a way that its amount is comprised in the range of from 15 g/L to 60 g/L.

Preferably, the inositol hexaphosphoric acid and salts thereof on the one hand, and the inositol on the other hand (preferably myo-inositol), are present in a mass ratio ranging from 1.5/1 to 8/1, preferably 2.5 to 6.

The pharmaceutical compositions comprising an anticancer compound according to the invention may contain a liquid or solid, for example aqueous, pharmaceutically acceptable excipient and/or vehicle. Many pharmaceutically acceptable excipients and/or vehicles can be used, for example solvents or diluents; water, where appropriate, mixed with propylene glycol or polyethylene glycol, buffered water, saline solution, a solution of glycine and derivatives thereof, a non-aqueous solution comprising especially solvents such as ethanol, N-methylpyrrolidone, dimethylacetamide (DMA), dimethyl sulfoxide (DMSO) and/or dimethylformamide (DMF), as well as agents required to reproduce the physiological conditions such as for example buffering agents and pH adjusters, surfactants such as Solutol® HS15, Tween® 80, sodium acetate, sodium lactate, sodium chloride, potassium chloride, calcium chloride, or a vehicle such as Cremophor EL®, this list being non-exhaustive. In addition, the pharmaceutical composition can be sterilized by sterilization techniques well known to those skilled in the art. It is preferred to use in the composition at least one aqueous solvent and/or at least one surfactant or alpha-hydroxylated carboxylic acid to solubilize the inositol hexaphosphoric acid, inositol pentaphosphoric acids, inositol tetraphosphoric acids or a salt thereof and magnesium chloride.

According to a preferred embodiment of the invention, the composition comprises an aqueous solvent for solubilizing the inositol hexaphosphoric acid, inositol pentaphosphoric acids, inositol tetraphosphoric acids or salts thereof, which is preferably combined with at least one alpha-hydroxylated carboxylic acid. According to a preferred embodiment, the liquid composition according to the invention is diluted in a green tea infusion at room temperature prior to the intake by the patient.

The alpha-hydroxylated carboxylic acid functions as both a solubilizing agent and a stabilizing agent. It prevents, at least partially, the phosphate moiety hydrolysis of the inositol hexa, penta or tetraphosphoric derivatives, resulting in liquid pharmaceutical compositions very stable over time and which can be highly concentrated in active principles.

The alpha-hydroxylated carboxylic acids suitable for use in the composition according to the present invention are described in detail in the patent EP 245277, on behalf of the applicant. In a preferred embodiment, the alpha-hydroxylated carboxylic acid is selected from the group consisting of glycolic acid, gluconic acid, glucuronic acid, tartaric acid (especially its enantiomers L and D, and its diastereoisomer meso), lactic acid, citric acid, malic acid and mixtures thereof.

Preferably, the alpha-hydroxylated carboxylic acid content of the composition corresponds to an amount of carboxyl functional group ranging from 3 mmol to 20 mmol per gram of phytic derivatives (phytic acid and phytin). This amount ensures optimum solubilization and stabilization of these compounds. The preferential amount of alpha-hydroxylated carboxylic acid to be supplied therefore varies according to the number of carboxyl moieties present on said alpha-hydroxylated carboxylic acid.

As pharmaceutically acceptable, non-toxic, inert vehicles, adjuvants or excipients, there can also be mentioned for illustrative purposes, but not limited to, solubilizing agents other than solvents, preservatives (in order to prevent especially bacterial or fungal contamination during packaging), wetting agents, emulsifiers, dispersing agents, binders, bulking agents, disintegrants, encapsulating agents, retardants, lubricants, absorbents, suspending agents, colorants, texturing agents, flavors, stabilizers, thickening agents, etc. Such compounds are for example magnesium carbonate, magnesium stearate, talc, lactose, pectin, dextrin, starch, gelatin, cellulosic materials, cocoa butter, etc.

Physiologically acceptable vehicles and excipients (or adjuvants) are also described in the book entitled “Handbook of Pharmaceutical Excipients,” Second edition, American Pharmaceutical Association, 1994.

Preferably, the mass of the dry extract of a liquid composition according to the invention represents from 10 to 30% of the total weight of this composition, more preferably from 15 to 25%. By dry extract, it is meant according to the present invention the remaining solids after evaporation of the solvents and volatile compounds in an oven at 100-110° C., optionally under vacuum.

Generally, the inositol hexaphosphoric acid, inositol pentaphosphoric acids, inositol tetraphosphoric acids and salts thereof represent from 25 to 55% of the mass of dry extract of the composition, and advantageously, from 30 to 50%.

Generally, the inositol hexaphosphoric acid and salts thereof represent from 25 to 55% of the mass of dry extract of the composition, and advantageously, from 30 to 50%, more preferably from 35 to 45%.

Generally, the magnesium chloride represents from 1 to 10% of the mass of dry extract of the composition, and advantageously, from 2 to 8%, more preferably from 3 to 7%.

Generally, the inositol (preferably myo-inositol) represents from 4 to 20% of the mass of dry extract of the composition, and advantageously, from 5 to 18%, more preferably from 7 to 15%.

Generally, the excipients represent from 25 to 55% of the mass of dry extract of the composition, and advantageously, from 30 to 50%, more preferably from 35 to 45%.

Generally, a pharmaceutical composition according to the invention comprises from 1% to 99% by weight, advantageously from 50% to 97% by weight, more preferably from 75 to 95% of an excipient and/or vehicle (or diluent) or a combination of pharmaceutically acceptable excipients and/or vehicles.

A pharmaceutical composition according to the invention is interchangeably in a solid form (generally water soluble powder, preferably in dry form, namely in the absence of solvent) or a liquid form. In liquid form, a pharmaceutical composition in the form of an aqueous suspension or aqueous solution will be preferred, preferably an aqueous solution, or in the form of a water-in-oil or oil-in-water emulsion or even a colloidal suspension in the presence of for example phospholipids, with a view to increasing bioavailability. Formulations wherein phytic compounds are in a solubilized state are preferred, but a formulation of the composition in powder form (for a final use preferably in liquid form) is also interesting because it prevents stability problems that may be encountered in liquid medium. Thus, the pharmaceutical composition can be especially packaged for example as a daily dose to be dissolved in an aqueous medium prior to the intake by the patient.

The preferred form for the composition according to the invention is aqueous solution, because it ensures a phytic compound bioavailability that is higher than solid formulations (capsule, tablet . . . ). By “aqueous solution” it is meant a solution comprising water as the main solvent, i.e. a solution comprising at least 50% by volume of water, relative to the total volume of said solution, preferably at least 90%. This aqueous solution may comprise from 0.1% to 10% by weight of a water-miscible solvent, especially ethanol or a polar aprotic solvent. In some embodiments, the aqueous solution comprises only water as solvent. The aqueous composition according to the invention has generally a pH ranging from 3 to 7.5, typically from 3 to 5.

The preparation of phytin aqueous solutions is described in detail in the patent EP 2452577, on behalf of the applicant.

In certain embodiments, the pharmaceutical composition is free from any texturing agent such as thickening agents and gelling agents. Indeed, the presence of these agents can be detrimental to the liquid nature and the homogeneity of the composition. In particular, the composition does not contain, in this embodiment, any gelling compounds such as agar-agar, starch, alginate salts, carrageenan and animal gelatins.

When a solid composition in tablet form is prepared, the main active ingredients are mixed with a pharmaceutical vehicle such as gelatin, starch, lactose, magnesium stearate, talc, acacia gum or the like.

Tablets can be coated with sucrose or other suitable raw materials or even can be treated in such a way that they have a sustained or delayed activity and continuously release a predetermined amount of active principle.

A capsule preparation is obtained by mixing active ingredients with a diluent and by pouring the resulting mixture in soft or hard capsules.

Active principles can also be formulated in microcapsule form, optionally with one or more carriers or additives.

Pharmaceutical composition in syrup or elixir form may contain active ingredients together with a sweetener, preferably calorie-free, methylparaben and propylparaben as antiseptics, as well as an agent adding flavor and a suitable colorant.

Water dispersible powders or granules may contain active ingredients in admixture with dispersing agents or wetting agents or suspending agents, such as polyvinylpyrrolidone, as well as with sweeteners or flavoring agents.

Generally, for the manufacture of a pharmaceutical composition according to the invention, the one skilled in the art can advantageously refers to the 9^{th} edition of the European Pharmacopeia issued in July 2016, or the last edition of the United States Pharmacopeia (U.S. Pharmacopeia, especially the USP 40-NF 35 edition).

Techniques for preparing pharmaceutical compositions according to the invention can be easily found by those skilled in the art, for example in the book Remington's Pharmaceutical Sciences, 17th edition, Mack Publishing Company, Easton, Pa., USA, 1985).

The pharmaceutical compositions according to the invention include more particularly those suitable for oral, topical, parenteral, nasal, intravenous, percutaneous (transcutaneous), subcutaneous, rectal, perlingual or respiratory administration, and especially simple or sugar-coated tablets, sublingual tablets, hard capsules, tablets, suppositories, creams, ointments, dermal gels, oral or injectable ampules or other liquid preparations.

The pharmaceutical composition of the present invention can be used for prophylactic and/or therapeutic administration. Thus, the active principles according to the present invention are prepared in a form suitable for the selected administration type, for example in liquid or lyophilized form.

For oral administration, a pharmaceutical composition according to the invention may be in the form of tablets, hard capsules, capsules, sugar-coated tablets, syrups, suspensions, solutions, powders, granules, emulsions, suspensions, microspheres or nanospheres, lipid vesicle suspensions or vesicles based on various polymers.

Specifically, for oral administration, a pharmaceutical composition according to the invention may be in the form of tablets which can be manufactured from solid compositions in combination with various excipients such as microcrystalline cellulose, sodium citrate, calcium carbonate, dicalcium phosphate, or glycine. Various disintegrating agents such as starch (from corn, potato, tapioca, etc.), alginic acid or even silicate can be used. Binding agents such as polyvinylpyrrolidone, sucrose, gelatin, or even acacia can be used. Lubricating agents such as magnesium stearate, sodium lauryl sulfate or even talc can be used. Such solid compositions as a powder can be used for manufacturing gelatin capsules. For solid compositions, lactose or even high molecular weight polyethylene glycol can be used.

For manufacturing liquid compositions for oral administration, inositol hexaphosphoric acid or a salt thereof and magnesium chloride can be combined with various sweetener agents, flavoring agents, coloring agents, optionally with also emulsifying agents or suspension agents, in combination with diluting agents such as water, ethanol, propylene glycol, glycerin or any combination of these excipient agents.

For parenteral administration, a pharmaceutical composition according to the invention may be in the form of solutions or suspensions for perfusion or injection. Thus, in particular, oil or water solutions or even suspensions, emulsions, or implants including suppositories, can be used. For example, the active principles of the composition can be dispersed in liquid vehicle such as a saline physiological liquid or even a saline solution containing 5% by weight of dextrose, that are conventionally used for the preparation of injectable pharmaceutical formulations.

For enteral administration, controlled release compositions can be used, for example compositions in which the active principles of the composition are protected from external environment by a plurality of coating layers which degrade in different ways, for example when in contact with a neutral or basic medium (gastro-resistant coating layers) or with a water medium (coating layers comprising soluble polymers or which degrade in water).

The dosage varies with the sex, age, weight and general condition of the patient, with the route of administration, cancer type, state of progress of the cancer, in particular, with whether metastases were detected or not in the patient. The dosage may also vary with the type of the related anticancer treatment(s). It is determined on a case-by-case basis, under medical supervision.

Generally, the inositol hexaphosphoric acid and salts thereof (and/or inositol pentaphosphoric acids, inositol tetraphosphoric acids and salts thereof) are used in amounts ranging preferably from 0.01 mg/kg of patient or animal body weight to 1 g/kg of patient or animal body weight per 24 hours, preferably from 5 to 500 mg/kg, in one or more doses, typically two daily doses. Preferably, said amount is at least equal to 10 mg/kg, more preferably 75 mg/kg. Preferably, said amount is at most equal to 500 mg/ kg, more preferably 250 mg/kg.

As an indication, in initial therapy, 6 to 8 g/day of inositol phosphoric derivatives according to the invention and 1.2 to 1.6 g/day of MgCl₂, to be taken in two divided doses between meals, can be provided; in supportive therapy to be maintained depending on the tumor marker analysis results, 3 to 4 g/day of inositol phosphoric derivatives according to the invention and 0.8 to 1.2 g/day of MgCl₂, to be taken as a single dose, can be provided; in primary or tertiary prevention treatment for decreasing the risk of relapse, 1 to 2 g/day of inositol phosphoric derivatives according to the invention and 0.4 to 0.8 g/day of MgCl₂, to be taken as a single dose, can be provided.

The composition according to the present invention can be packaged in a multi-dose container or in a single-dose container. Preferably, it is packaged in dose units, for example in the form of doses or scored single-dose ampules.

As already mentioned previously in the present description, the composition according to the invention has an antiproliferative-type but non-cytotoxic activity towards cancer cells, from which was derived a very important benefit which is the absence or the virtual absence of side effects of a treatment by means of said composition. Indeed, its components are not toxic and might lead to at most a risk of anemia in the long term due to the iron complexation by the phosphoric derivatives of inositol.

The precise mechanism of action of a composition according to the invention is not known. Without being bound by any theory, it is possible that the magnesium chloride acts as anti-inflammatory or a non-specific immune defense activator, and that the inositol phosphoric compounds relieve the apoptosis inhibition and restrain cancer cell angiogenesis. Also, the cancer cell redifferentiation and back to the normal phenotype might be involved, as seen on plant cell cultures carried out in the presence of phytic acid (Parc G., Rembur J., Rech P., Chriqui D., Plant Cell Rep. 2007, 26, 145-52). These inositol phosphoric compounds could also operate by plasma iron sequestration or by neutralizing compounds essential for tumor progress such as polyamines.

The effects of the composition according to the invention result from a synergy between magnesium chloride and inositol phosphoric compounds or salts thereof (see experimental section). The magnesium chloride has no antitumor effect. It was initially used during the First World War by the Professor Pierre Delbet as an antiseptic for disinfecting wounds of war. Until the 1960s, it was successfully used for treating bacterial or viral infections as severe as diphtheria or poliomyelitis.

Contrary to the monoclonal antibodies against specific targets (defective genes or tumor type specific tumor antigens), the composition according to the invention, by the nature of its components, is effective on many tumor types.

In the present application, cancer cells refer to cells having characteristics typical of cells causing cancer, such as uncontrolled proliferation, immortality, metastatic potential, rapid growth and high proliferation rate, and some specific morphological features. Cancer cells are often in the form of a tumor, but such cells can occur alone inside the body, or may be non-tumorigenic cancer cells, such as leukemic cells. Cancer cells can be related to many types of cancers, including but not limited to, leukemia, periampullary cancer, lymphoma, melanoma, neuroblastoma, liver, ovarian, brain, lung (in particular non-small-cell lung cancer), colon, breast, pancreatic, prostate, testicular, esophageal, gallbladder, small intestine, uterine, cervical, renal, stomach, bladder, cerebrospinal, colorectal cancer, glioblastoma multiforme, preferably pancreatic cancer. The pharmaceutical compositions of the invention can be used for the therapeutic treatment of at least one of the above mentioned cancers, including a cancer selected from breast cancer, periampullary cancer, pancreatic cancer, liver cancer, gallbladder cancer, small intestine cancer and colorectal cancer.

The object of the invention is therefore a pharmaceutical composition as defined previously, intended to be used for therapeutic purposes, i.e. as a drug for human or veterinary use, especially, for use in the treatment and/or the prevention of a metastatic or primary cancer.

The invention also relates to a therapeutic treatment method for preventing and/or treating cancer development in a subject or patient in need thereof, said method comprising a step during which a therapeutically effective amount of a composition as defined in the present description is administered to the patient, either alone or in admixture with one or more pharmaceutically acceptable excipients and/or vehicles.

In the present description, a patient refers to both a human and an animal, especially a non-human mammal.

The invention can be implemented in combination with other treatments modalities, such as hormonotherapy, surgery, cryotherapy, hyperthermia, radiotherapy, additional chemotherapy, etc. Advantageously, the treatment according to the invention can be combined with another treatment targeting the cancer cell proliferation and/or angiogenesis.

The invention is further illustrated, but is not limited to, by the following examples.

### EXPERIMENTAL SECTION

The mass composition of the phytin used, provided by Tsuno Fine Rice Chemicals (Wakayama, Japan), is as follows: phosphorus 23.3%, calcium 18.5%, magnesium 4.8%. The anhydrous magnesium chloride, myo-inositol and citric acid monohydrate are of Codex grade and provided by Coger (79 rue des Morilions 75015 Paris).

A composition according to the invention was administered to a voluntary patient who developed a cancerous tumor and demonstrated its in vivo antitumor effectiveness.

1. Preliminary Studies

Acute toxicity: Phytin solutions have no visible side effects upon oral administration at a single dose of 2240 g/kg in male and female rat of the Wistar line. The lack of toxicity of the magnesium chloride in man is already known.

Antiproliferative activity of phytin solutions: Tests on cancer cell strains were carried out on the following strains: DU-145 (human prostatic cancer), EMT-6 (mouse mammary carcinoma), SW-480 (human colon adenocarcinoma), MCF-7 (ER-positive mammary tumor human line). The LD50 concentrations on these strains are between 250 and 500 μM. These concentrations are higher than those of the conventional antitumor agents (doxorubicine, etoposide, methotrexate, 5-fluorouracil), but it is important to note that phytin solutions are not toxic.

2. Patient and Treatment

The patient is a 90-year-old man with a history of benign overgrowth of the prostate, dyslipidemia, high blood pressure, cardiac arrhythmia and atrial fibrillation and basal cell carcinoma. His general condition deteriorated with severe fatigue, digestive disorders, sarcopenia and weight loss of 6 kg during the last three months prior his admission to emergency department with jaundice subsequent to abdominal pains, nausea, vomiting and syncope.

Before his admission to emergency department, the patient consumed, on a regular basis, with a view to prevention and for 5 years as an oral food supplement, 1.4 g/day of phytin solubilized in citric acid.

A scanner showed a dilatation of the biliary tract and the duct of Wirsung. An echo-endoscopy confirmed the presence of intra-ampullary development periampullary cancer (malignant tumor of the hepatopancreatic ampulla (ampulla of Vater)) of neoplastic etiology. The usual solution for this type of pathology is a cephalic pancreaticoduodenectomy (surgical ablation of the head of pancreas, of the entire duodenum, the gallbladder and often of the bottom third of the stomach).

The patient was treated during 66 days by means of two daily doses of treatment according to the invention, in the morning and the afternoon between meals.

The preparation of one treatment dose is carried out as follows: a mixture comprised of 3.2 g of phytin and 0.88 g of myo-inositol is dispersed in 30 mL of water. 3.4 g of citric acid monohydrate are added in order to dissolve the phytin, as well as a 392 mg tablet of anhydrous magnesium chloride (that is 100 mg of magnesium). Upon complete dissolution, this preparation is diluted with a green tea infusion cooled down before being ingested at room temperature.

3. Clinical Trial Results

At the end of the 66 day treatment, a ¹⁸FDG scintigraphy was carried out (Positron Emission Tomography—PET-SCAN—on Siemens Biograph mCTFLOW, injection of 137MBq of ¹⁸FDG, PET acquisition coupled to a low dose attenuation correction computed axial tomography without contrast medium injection). It indicates a lack of focus discernible from background in the pancreas and the duodenopancreatic confluence. There is no basis either for a secondary lymph node, visceral or bone involvement.

A hepatic MRI scan and cholangio-MRI carried out four days later indicate a lack of expanding lesion in the cephalic pancreas and at ampullar level.

An echo-endoscopy performed under general anaesthesia 40 days later using the echo-endoscopy probe Olympus confirms that the periampullary cancer disappeared.

The fact that the treatment according to the invention is active against pancreas cancer, one of those cancers, the prognosis for life of which is the poorest, suggests that this treatment should be effective against many types of cancers. Pancreatic cancer is a highly aggressive form of cancer, which is resistant to all chemotherapies. The survival after the pancreatic cancer detection does not generally exceed 5 months.

These outstanding clinical outcomes were totally unexpected in view of the known properties of the phytic derivatives on the one hand and of the magnesium chloride on the other, and are clearly due to an in vivo synergy. Indeed, the effect resulting from their association is not commensurate with the sum of the effects seen when they operate independently.

On the one hand, the fact that the patient regularly consumed phytin prior the periampullary cancer diagnostic confirms that the phytin does not have antitumor activity in human when used in the absence of magnesium chloride.

On the other hand, magnesium derivatives have never been successful in treating cancer. In that respect, Doctors J. Sal and Y. Donadieu stated: “It is perfectly obvious that it is impossible to treat a cancer of any kind by means of a treatment only comprising magnesium species” (Le Magnésium: thérapeutique naturelle, Ed. Maloine, Paris, 1986, p. 66).

These results have to be matched up with the following comparative data, available in the literature. Phytin and inositol, used as a supplement to chemotherapy, at the same dose as that of the treatment implemented above but in the absence of magnesium chloride, entail only a reduction of the side effects (Journal of Experimental & Clinical Cancer Research 2010, 29: 12). The inventor also noted that in vitro, magnesium chloride did not improve the antitumor activity of the phytin solubilized by citric acid on the mammary tumor cell line MCF-7 (no change in the median inhibitory concentration IC₅₀), and that myo-inositol had no in vitro antitumor action on the MCF-7 strain.

1. A therapeutic method of treating and/or preventing cancer comprising administering to a subject in need thereof an effective amount of a pharmaceutical composition comprising:
at least one compound selected from inositol hexaphosphoric acid, inositol pentaphosphoric acids, inositol tetraphosphoric acids or one of their salts, and
magnesium chloride,
in combination with one or more pharmaceutically acceptable excipients and/or vehicles.

2. The therapeutic method of claim 1, wherein the pharmaceutical composition comprises inositol hexaphosphoric acid or a salt thereof, present in the form of its stereoisomer myo-inositol hexaphosphoric acid or a salt thereof.

3. The therapeutic method of claim 1, wherein the pharmaceutical composition comprises inositol hexaphosphoric acid, present in the form of a mixed salt of calcium and magnesium.

4. The therapeutic method of claim 1, wherein the pharmaceutical composition further comprises inositol.

5. The therapeutic method of claim 4, wherein inositol hexaphosphoric acid, inositol pentaphosphoric acids, inositol tetraphosphoric acids and salts thereof on the one hand, and inositol on the other hand, are present in the pharmaceutical composition in a mass ratio ranging from 1.5/1 to 8/1.

6. The therapeutic method of claim 1, wherein the pharmaceutical composition further comprises at least one alpha-hydroxylated carboxylic acid.

7. The therapeutic method of claim 6, wherein the alpha-hydroxylated carboxylic acid is selected from lactic acid, tartaric acid, glycolic acid, gluconic acid, glucuronic acid, citric acid and malic acid.

8. The therapeutic method of claim 1, wherein the pharmaceutical composition further comprises an aqueous solvent.

9. The therapeutic method of claim 1, wherein the pharmaceutical composition is in the form of an aqueous solution, inositol hexaphosphoric acid, inositol pentaphosphoric acids, inositol tetraphosphoric acids and salts thereof being present in an amount of from 70 g/L to 270 g/L.

10. The therapeutic method of claim 1, wherein the pharmaceutical composition is in the form of a powder.

11. The therapeutic method of claim 1, wherein inositol hexaphosphoric acid, inositol pentaphosphoric acids, inositol tetraphosphoric acids and salts thereof on the one hand, and magnesium chloride on the other hand, are present in the pharmaceutical composition in a mass ratio ranging from 2/1 to 16/1.

12. The therapeutic method of claim 1, wherein the pharmaceutical composition further comprises myo-inositol.

13. The therapeutic method of claim 1, wherein the cancer is selected from cancer selected from breast cancer, periampullary cancer, pancreatic cancer, liver cancer, gallbladder cancer, small intestine cancer and colorectal cancer.

14. The therapeutic method of claim 1, wherein the cancer is pancreas cancer.
